(19) United States
(12) Patent Application Publication
(10) Pub. No.: US 2024/0398723 A1
(43) Pub. Date: Dec. 5, 2024

(54) METHOD FOR MANUFACTURING A MEDICAL PATCH FOR LOCAL AND CONTROLLED RELEASE OF BIOACTIVE SUBSTANCES FOR THE TREATMENT OF CHRONIC ULCERS, AND MEDICAL PATCH ACHIEVED WITH SUCH METHOD

(71) Applicant: CONSIGLIO NAZIONALE DELLE RICERCHE, Roma (IT)

(72) Inventors: SOLDANI Giorgio, Massa (IT); Paola LOSI, Massa (IT); Tamer ALKAYAL, Avenza (Massa-Carrara) (IT); llenia FOFFA, Massa (IT); Aida CAVALLO, Pisa (IT); Marianna BUSCEMI, La Spezia (IT)

(21) Appl. No.: 18/699,585

(22) PCT Filed: Oct. 7, 2022
(86) PCT No.: PCT/IB22/59601

§ 371 Date: Apr. 8, 2024

(30) Foreign Application Priority Data
Oct. 7, 2021 (IT) 102021000025664

(51) Int. Cl. A61K 9/70; A61K 35/19; A61K 38/36; A61K 38/48; B29C 41/08; B29C 41/46; B29K 105/00; B29L 31/00

(52) U.S. Cl. CPC A61K 9/7023; A61K 35/19; A61K 38/363; A61K 38/4833; A61K 38/484; B29C 41/085; B29C 41/46; B29K 2089/00; B29K 2105/0035; B29L 2031/753

(57) ABSTRACT

A method for manufacturing a medical patch for the treatment of chronic vascular and diabetic ulcers comprises the steps of preparing a tubular support, spraying toward the axial lateral surface of said support at least two separate, simultaneous, converging jets of two nebulized solutions containing fibrinogen and thrombin, respectively, rotating the support and/or orienting the jets in such a way as to deposit on said support a layer of material (M) of predetermined size, and incubating the material (M) until the fibrin contained therein is polymerized.

### TECHNICAL FIELD

The present invention is generally in the field of pharmaceutical preparations; in particular, the invention relates to a method for manufacturing a medical patch for the local and controlled release of bioactive substances for the treatment of tissue lesions (e.g., chronic, vascular, and diabetic ulcers), and to a medical patch obtained by such a method.

### SUMMARY OF THE INVENTION

Chronic skin lesions may be debilitating and painful for patients, increase the risk of mor-bidity and mortality, and represent a major financial burden on health systems.

The treatment of chronic wounds has attracted a great deal of interest in recent years, both scientifically and economically, from biomedical firms that are investing in research into new therapies. Multiple approaches for the treatment of chronic lesions have been investi-gated, yet at this time their treatment is still a challenge.

The use of innovative materials for the treatment of chronic skin lesions has recently gained increasing interest. A particularly relevant aspect is chronic lower limb ulcers. These are wounds that may originate from a variety of pathologies, but mainly result from pathological conditions such as critical ischemia of the lower limbs (CLI) and diabetes. CLI is a chronic peripheral arterial disease characterized by the presence of pain at rest and/or trophic lesions, such as ulcers and gangrene.

The incidence of CLI is very high. The importance of this phenomenon is therefore extremely significant both in terms of the number of patients involved and the time and resources required to treat them.

In the past 15 years, the number of patients with diabetes has increased 6-fold. Estimates for 2030 for the world diabetic population speak of 370 million sufferers—a true epidemic for a disease that has fearsome complications and very high social and healthcare costs.

The most feared and disabling complication of this chronic disease is the so-called “diabetic foot,” which, if not prevented and treated, inevitably leads to amputation of the limb.

The incidence of new ulcers is about 2/100 diabetics/year, compared with an expected incidence in the general population of 2/1,000/year. The importance of this phenomenon is therefore extremely significant both in terms of the number of patients involved and the time and resources required to treat them.

To manage this complication and thus reduce the significant socioeconomic impact, appro-priate treatments are needed to increase the patient's overall quality of life. Because the vas-culature in this type of disease may be severely impaired and possibly induce tissue necrosis, the development of a therapy that is able to promote the formation of new collateral vessels could enable faster and more complete healing.

To date, several strategies have been developed consisting mainly of the injection of recom-binant growth factors into the wound site.

Unfortunately, direct application of factors to the site of the lesion is not very effective because they spread rapidly from the injection site and are enzymatically digested or inacti-vated. Furthermore, topical nanoparticles present in solution or powder form may rapidly diffuse from the wound, thus requiring daily administration.

Different types of wound dressings are also commercially available, differing in their mode of application, materials, shape, and methods used in their production. Generally, these dressings are inert and are made of synthetic materials such as polyurethane or natural materials such as sodium carboxymethyl cellulose hydrocolloid fibers (Hydrofiber).

Recently, special emphasis has been placed on systems employing biomaterials and on the latest and most innovative therapeutic strategies and delivery systems.

An example of a solution of the aforesaid type is known from document EP 2435099 B1, in which a fibrin layer supported by a layer of a synthetic material (specifically, polyurethane) is described. In this case, the polyurethane layer is introduced because the fibrin itself is mechanically weak, and therefore needs support in order to be applied. Thrombin is adsorbed onto the polyurethane layer, and the crosslinking of the fibrinogen to fibrin is achieved on the polyurethane support according to the traditional method, that is, according to the fibrin-ogen/thrombin reaction in the presence of calcium ions. In other words, the fibrin gel is formed locally on the polyurethane support, which thus imparts mechanical strength and elasticity to the construction.

In any case, the materials to be used for making patches for the treatment of chronic wounds should facilitate tissue regeneration, restore tissue function, and promote a rapid healing process through the release of bioactive factors. Furthermore, in the case of a biodegradable material, it should possess a degradation rate that matches the tissue growth rate, and neither the material nor the byproducts of the degradation process should induce immunogenicity and toxicity.

However, the fibrin obtained by the classical method of mixing fibrinogen with thrombin in the presence of calcium ions, leads to the formation of a gel lacking any mechanical strength.

The prior art contemplates some alternative solutions to simply mixing fibrinogen with thrombin, particularly by deposition on a support of solutions sprayed by means of spray nozzles.

For example, U.S. Pat. No. 6,074,663 A describes a process for preparing a self-supporting crosslinked fibrin matrix obtained through the simultaneous mixing of two streams of solutions containing fibrinogen and thrombin, respectively. However, because of the rapid crosslinking of the fibrin, the solution resulting from the simultaneous mixing of fibrinogen and thrombin is not suitable for delivery by a spray nozzle, as the density and viscosity of the dispensed material would tend to easily choke the nozzle.

Alternatively, U.S. Pat. No. 7,759,082 B2 discloses a process in which the two solutions are delivered separately, using nozzles with parallel axes. Although this procedure solves the problem of nozzle choking, it does not lend itself to making a patch with optimal mechanical features, as the matrix deposited on the support will suffer from poor consistency due to the dispersion of the jets, which will have only a narrow (and difficult to control) area of mutual overlap. In fact, the two delivery cones will interfere with each other only partially, creating frayed and uneven crosslinking zones, to the detriment of the mechanical features of the patch. Based on these considerations, the invention provides, for overcoming the above limitations, the administration of medical substances through the use of an improved, self-supporting elastic fibrin matrix of such mechanical consistency that it may be easily cut out and applied to a wound, allowing for the eventual local and controlled release of bioactive factors at the site of the lesion.

The invention may also provide for the local administration of “bioactive” substances through the application to the lesion of porous matrices based on natural and/or synthetic polymers that incorporate the bioactive substances and allow them to be released in a gradual and controlled manner over time.

The technical problem that the invention solves concerns the preparation of an advanced dressing based on a crosslinked fibrin matrix that is obtained by a multi-way spray deposition process and in which a fibrinogen solution and a thrombin solution (and possibly an additional solution containing the “bioactive” factors) are sprayed separately, simultaneously, and convergently over a cylindrical substrate.

The convergence of the flows (i.e., the feature whereby the axes of the dispensing nozzles are incident to each other) allows the two solutions to be focused in a defined and circum-scribed region, which greatly improves the mechanical and morphological features of the matrix, with respect, for example, to the known case with parallel nozzle axes.

In addition, additional bioactive factors, e.g., platelet lysate (preferably obtained from umbilical cord blood) and/or plasminogen, may also be effectively loaded into the dressing at the time of manufacture.

The resulting bioactive dressing is able to rapidly stimulate healing of diabetic ulcers, as demonstrated by in vivo experiments in diabetic mice. It has also been shown by in vivo experiments on fibroblast and keratinocyte cell cultures that the platelet lysate and plasminogen, when present simultaneously in the fibrin matrix, have a synergistic action on cell replication and migration, further enhancing it with respect to the separate presence of the factors in the fibrin matrix.

The main advantage of the described method using two separate pathways and the simultaneous and convergent deposition of fibrinogen and thrombin (ideally sprayed at low flow, so as to avoid dripping phenomena with consequent loss of material) is to allow crosslinking (polymerization) of fibrinogen (which turns into fibrin) in thin, sequential layers until a given thickness of fibrin is formed.

All the intimately crosslinked layers contribute to the formation of a fibrin patch with improved properties that is elastic, mechanically strong, and self-supporting, and which may be easily applied and adapted to a wound like any dressing. Furthermore, during the manufacturing process using the multi-jet spray technique, bioactive substances of various kinds, preferably plasminogen and/or platelet lysate obtained from cord blood or peripheral blood, may be incorporated into the fibrin matrix being formed together with or alternatively to metalloprotease inhibitors and/or antibiotics and/or anti-inflammatoires and/or either synthetic or biological nanoparticles loaded with active ingredients of various kinds and/or nanovesicles secreted by various cell types (called “exosomes”) and containing bioactive molecules (e.g.: proteins, lipids, nucleic acids, etc.). All these bioactive substances may be easily incorporated into the fibrin patch at the time of its manufacture.

Another interesting feature of a patch made according to the present invention is that said patch, once prepared, may be freeze-dried (locked) in its present state, packaged, and subsequently rehydrated with saline solution at the time of its application to the wound. Once rehydrated, the patch begins to release the bioactive substances locally for several days until it is completely degraded, thus acting as a biodegradable system for controlled-release of these factors.

An additional advantage of the present invention is that of developing a method for creating a dressing that, upon manufacture, may be directly loaded with bioactive substances that may stimulate the rapid healing of chronic diabetic and vascular ulcers that are otherwise untreatable with conventional dressings. The use, for example, of platelet lysate from cord blood, which has a growth factor content 4-5 times higher than in adults and an immunologic immaturity that makes the likelihood of adverse reactions very limited, represents an oppor-tunity of considerable therapeutic interest at low cost. Said product, which is in fact a waste product, is accessible because, in umbilical cord blood banks, up to 85 percent of the samples are unsuitable for hematopoietic transplantation because of a reduced level of cells and may therefore be used for non-transfusion use, overcoming the difficulties associated with the use of autologous platelet lysate, which requires taking more samples from patients with chronic lesions.

The aforesaid and other objects and advantages are achieved, according to one aspect of the invention, by a method for manufacturing a medical patch for the local and controlled release of bioactive substances for the treatment of chronic lesions (ulcers), and a medical patch obtained by such a method, having the features defined in the appended claims. Preferred embodiments of the invention are defined in the dependent claims.

### BRIEF DESCRIPTION OF THE DRAWINGS

The functional and structural features of some preferred embodiments of a method for manufacturing a medical patch according to the invention will now be described. Reference is made to the accompanying drawings, in which:

FIG. 1A is a schematic perspective view of an apparatus for manufacturing a medical patch for the local and controlled release of bioactive substances, according to an embodiment of the present invention;

FIG. 1B a), b), c) are schematic views of three stages of manufacturing a medical patch according to an embodiment of the present invention;

FIGS. 2A and 2B are, respectively, a schematic view of a pair of nozzles with converging axis showing the deposition angle and velocity vectors in a specific embodiment in which there is a combination of a rotational motion of the mandrel and a translation of the nozzles parallel to the axis of the mandrel, and a perspective schematic view of an apparatus comprising a pair of nozzles with converging axis, supported by a movable carriage, according to an embodiment of the invention;

FIGS. 2C and 2D are two schematic views of respective deposition footprints, illustra-tively indicative of the incidence zones of the flows on a support when the nozzles have parallel axes, as in the prior art, and incident axes, as in the present invention, respectively;

FIG. 3 is a stress-strain diagram related to a plurality of patch samples subjected to tensile testing;

FIG. 4 is a schematic diagram of the time scan of the monitoring steps of the dressing applied to animals undergoing in vivo testing;

FIG. 5 is a comparative representation of lesions induced on different specimens and treated with different modalities, photographed at three moments of the aforesaid experimentation; and

FIG. 6 is an illustrative diagram of the percentage of open wound area at 7 and 14 days under the aforesaid test conditions.

### DETAILED DESCRIPTION

Before explaining in detail a plurality of embodiments of the invention, it should be clarified that the invention is not limited in its application to the design details and configuration of the components presented in the following description or illustrated in the drawings. The invention may assume other embodiments and be implemented or constructed in practice in different ways. It should also be understood that the phraseology and terminology have a descriptive purpose and should not be construed as limiting.

A method for manufacturing a medical patch for the local and controlled release of bioactive substances for the treatment of vascular and diabetic ulcers according to the invention comprises the steps of arranging a mandrel 10, in the form of a tubular support; spraying at least two separate, simultaneous, and converging jets of a nebulized solution containing fibrinogen, and a nebulized solution containing thrombin, respectively, toward the axial lateral surface of said mandrel 10.

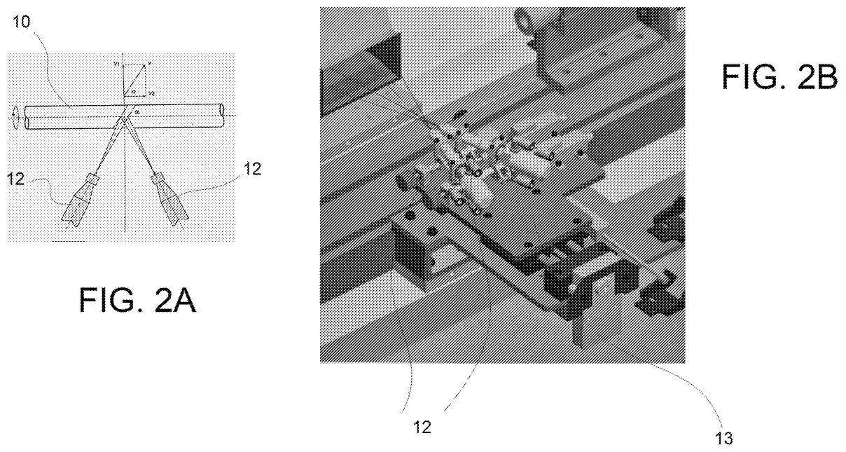

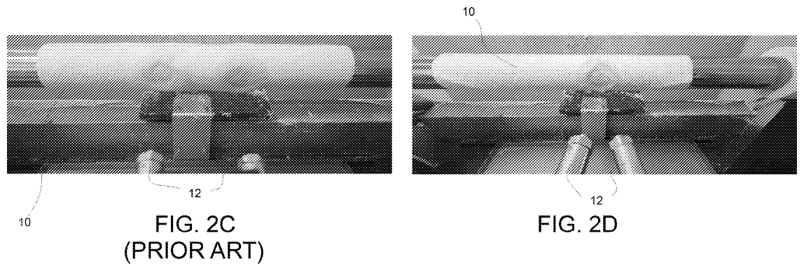

The expression “converging jets” means spray jets having axes that are mutually incident. According to one embodiment, the point of convergence of the axes of the jets may be on the outer surface of the mandrel 10, or (as shown by way of example in FIG. 2A) at the axis of the mandrel 10, or at a point outside the mandrel 10. Shape, size, consistency, and weave of the resulting matrix may depend on, and be varied by, the position of the point of convergence of the axes of the jets.

As may be seen from FIGS. 2C and 2D, the overlap areas of the jets, in cases with parallel and converging axes, differ significantly. In fact, the two images show two patterns of deposition impressions obtained by spraying two solutions separately on a paper substrate wrapped on a stationary mandrel, and it may be easily observed that, in the pattern produced by the parallel jets, the impressions are spaced and distinguishable, with an intermediate area sparsely affected by deposition where the two jets interacted weakly. This results in a very thin and fragile fibrin patch.

On the other hand, in the case of converging jets (FIG. 2D), said dispensers used for the previous test were rotated until the jets were concentrated on a limited area of the mandrel, and a sharp, deep imprint is indeed visible. This deposition pattern achieves a complete overlapping of the jets, which in practice merge into a single stream focused on a relatively cir-cumscribed area of the mandrel, thus allowing instantaneous formation of the fibrin gel, with improved physical consistency. In fact, the fibrin patch thus obtained demonstrates optimal handling features and mechanical strength, and is significantly superior to those achieved with the deposition pattern produced by the parallel jets of the prior art.

The step is also provided of rotating the mandrel 10 about its axis and/or orienting the aforesaid jets so as to strike a predetermined circumferential portion of the axial lateral surface of the mandrel 10, until a layer of material M of predetermined size is deposited on that circumferential portion. In the example shown, the portion of the lateral surface of the mandrel 10 struck by the jets extends angularly along the entire circumferential arc of the mandrel 10, so that the material deposited on the mandrel 10 forms a closed annular (or tubular) structure.

By virtue of the interaction between fibrinogen and thrombin, the material M deposited on the mandrel 10 polymerizes to form sequential layers of crosslinked fibrin.

Said material M is subsequently incubated (preferably, at 37° C.) until complete polymeriza-tion/crosslinking of the fibrin contained therein (“curing” phase).

According to a preferred embodiment, the step of striking a predetermined circumferential portion of the axial lateral surface of the mandrel 10 is implemented by spraying toward that axial lateral surface of the mandrel 10 a third separate jet, simultaneous with and convergent to the other two (with respective nebulized solutions containing fibrinogen and thrombin), of a nebulized solution containing plasminogen (PLG) and/or platelet lysate (PL) and/or metalloprotease inhibitors and/or antibiotics and/or anti-inflammatoires, and/or nanoparticles loaded with active ingredients and/or nanovesicles secreted from cells and containing bioactive molecules.

Preferably, the platelet lysate (PL) is a platelet lysate obtained from cord blood (CB-PL).

According to one embodiment, the fibrinogen-containing solution contains fibrinogen in a concentration between 20 mg/ml and 100 mg/ml, and the thrombin-containing solution contains thrombin in a concentration between 500 IU/ml and 3500 IU/ml.

Preferably, the solution containing platelet lysate (PL) contains platelet lysate (PL) in a concentration between 0.5×10⁹ plt/ml and 10×10⁹ plt/ml and the solution containing plasminogen (PLG) contains PLG in a concentration between 5 mg/ml and 100 mg/ml.

Expediently, the step of striking the axial lateral surface of the mandrel 10 with the jets of nebulized solution is implemented by means of dispensers 12, appropriately configured as common spray guns equipped with end nozzles from which a stream of atomized fluid exits. The dispensers 12 are oriented so that their axes are mutually incident. According to one embodiment, the point of convergence of the axes of the dispensers 12 may be on the outside surface of the mandrel 10, or at the axis of the mandrel 10, or at a point outside the mandrel 10.

Preferably, the nebulized solution containing fibrinogen is dispensed from the respective dispenser 12 with a flow rate between 0.1 ml/min and 0.4 ml/min, and nebulized solution containing thrombin is delivered from the respective dispenser 12 with a flow rate between 0.05 ml/min and 0.4 ml/min. According to one embodiment, the nebulized solution containing platelet lysate (PL) and/or plasminogen (PLG) is also dispensed by the respective dispenser 12 with a flow rate between 0.05 ml/min and 0.4 ml/min.

According to one embodiment, the step of rotating the mandrel 10 about its own axis and/or orienting the jets such that they strike a predetermined circumferential portion of the axial lateral surface of the mandrel 10 is implemented by translating the dispensers 12 along a direction parallel to the axis of the mandrel 10 and/or rotating the dispensers 12 about the axis of the mandrel 10. As an example, the dispensers 12 may be carried by a movable carriage 13 along a straight and/or circular direction, said circular direction extending about the axis of the mandrel 10.

The possible presence of a combined rotational motion of the mandrel 10 (about its own axis) and translation of the dispensers 12 (along a direction parallel to the axis of the mandrel 10) in a convergent configuration proves to be particularly advantageous, as it gives peculiar morphological properties to the fibrin matrix. As illustrated by way of example in FIG. 2A, an angle of deposition α of the material M (obtained from the superposition of the converging flows of fibrinogen and thrombin) on the mandrel 10 may be determined by varying a tan-gential velocity vector V1, dependent on the rotational speed of the mandrel 10, and/or a linear velocity vector V2, dependent on the translation speed of the dispensers 12 parallel to the axis of the mandrel 10. As a function of these velocities, the deposition angle α will be substantially defined by the following relationship:

$$tan α = V 1 / V 2$$

The possibility of varying this angle of deposition α, by varying the aforesaid speeds V1, V2, and/or the distance of the dispensers 12 from the mandrel 10 and the angle of convergence of said dispensers 12 (such that, for example, the jets are brought substantially to fully overlap directly on the outer surface of the mandrel 12) allows the material M to be wound precisely spirally about the mandrel 10 and, through the reciprocating translating motion of the dispensers 12, to compose a woven pattern for the fibrin matrix characterized by excel-lent isotropic mechanical properties (e.g., when V1=V2, therefore with α=) 45°.

Preferably, the outer diameter of the mandrel 10 is within a range of 3-10 cm, and/or the rotational speed of the mandrel 10 is within a range of 30-120 rpm, and/or the translation speed of the dispensers 12 along a direction parallel to the axis of the mandrel 10 is within a range of 10-50 cm/s, and/or the extension in the axial direction of the portion of the mandrel 10 struck by the overlapping jets is within a range of 2-20 cm, and/or the distance of the outlet orifice of the dispensers 12 from the axial lateral surface of the mandrel 10 is within a range of 2-6 cm, and/or the supply pressure of air to the dispensers 12 to generate the jets is within a range of 8-16 psi.

According to an embodiment, the method comprises the step of subjecting the obtained patch to freeze-drying after the polymerization step of the material deposited on the mandrel (M). For example, the freeze-drying procedure may involve the extraction of water under vacuum after freezing the material at −50° C.

Preferably, the freeze-drying step is preceded by the step of fixing the layer of polymerized material M on a plastic support, and the step of freezing the resulting assembly at −50° for 30 min.

According to one aspect of the invention, a medical patch is provided for the local and controlled release of bioactive substances obtained according to any of the embodiments of the method described above.

Expediently, such a medical patch is suitable for use in the therapeutic treatment of chronic vascular and diabetic ulcers.

The medical patch according to the invention may also lend itself for use as a filler for the treatment of tissue defects, i.e., for the restoration of at least partial mechanical continuity of a damaged tissue (e.g., ulcerated or degraded dental or skin tissue or a tissue cavity formed as a result of tumor removal, etc.). In such an application, the patch would, for example, be used to fill tissue defects and stimulate tissue regeneration in the area of the defect. Being biodegradable, it is then supplemented and replaced by newly formed tissue.

According to a further aspect of the invention, an apparatus for producing a medical patch comprises a mandrel 10, in the form of a tubular support (in the illustrated example, with a horizontal axis), and a plurality of dispensers (12), each configured to dispense a nebulized solution in such a way that the outflowing jet strikes at least part of the axial lateral surface of said mandrel (10).

The mandrel 10 is rotatable about its own axis, and/or the dispensers 12 are translatable along a direction parallel to the axis of the mandrel 10 and/or rotatable about the axis of the mandrel 10.

Preferably, the dispensers 12 are configured to each dispense a flow rate of nebulized solution between 0.05 ml/min and 0.4 ml/min.

According to an embodiment, the diameter of the mandrel 10 is within a range of 3-10 cm, and/or the mandrel 10 is configured to rotate with a speed within a range of 30-120 rpm, and/or the dispensers 12 are configured to translate along a direction parallel to the axis of the mandrel 10 with a speed within a range of 10-50 cm/s, and/or the dispensers 12 are configured to jointly strike with their respective jets a portion of the mandrel 10 extending in the axial direction within a range of 2-20 cm, and/or the distance of the outlet orifice of the dispensers 12 from the axial lateral surface of the mandrel 10 is within a range of 2-6 cm, and/or the dispensers 12 are configured to be supplied with air at a pressure within a range of 8-16 psi.

Throughout this description and in the claims, the terms and expressions indicating positions and orientations, such as “axial,” “transverse,” etc., refer to the axis of the mandrel 10.

### Experimental Validation

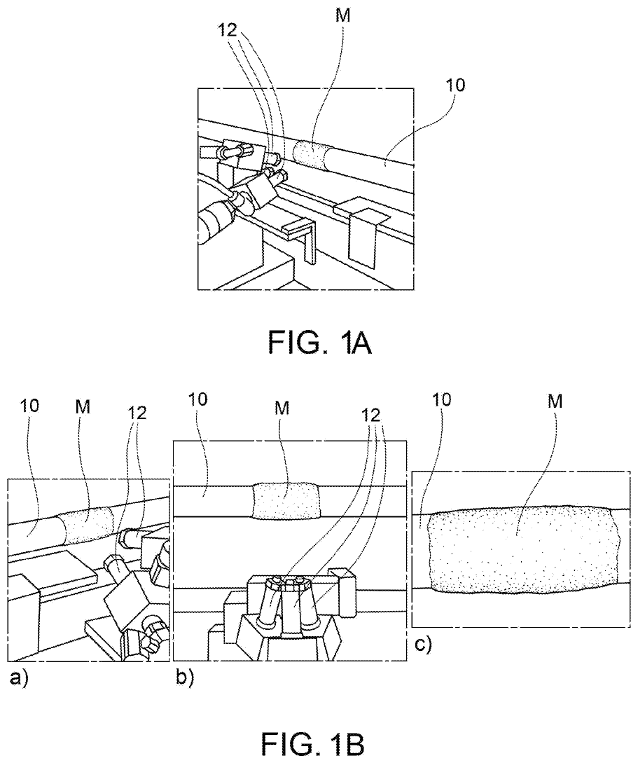

Patches according to the invention were produced by means of an apparatus equipped with a system of three converging spray guns that allows the solute contained in three different solutions to be deposited by separate and simultaneous sprays on a rotating cylindrical mandrel (FIG. 1). With this technology, it was possible to achieve the formation of a consistent layer of crosslinked (in other words, mechanically resistant) fibrin on a rotating mandrel.

Specifically, bioactive fibrin-based medical patches were produced using the three guns containing the following solutions, respectively:
spray gun 1: aqueous solution of fibrinogen;
spray gun 2: aqueous solution of thrombin;
spray gun 3: aqueous solution of plasminogen (PLG) and platelet lysate from cord blood (CB-PL).

Freeze-dried fibrinogen and thrombin were solubilized in their respective solvents: fibrinogen at 37° C., thrombin at room temperature.

The following solutions were prepared:
fibrinogen: 480 mg of fibrinogen in 8 ml of solvent (60 mg/ml);
thrombin: 2500 IU of thrombin in 2 ml of solvent (1250 IU/ml);
CB-PL (5×10⁹ plt/ml) and PLG (5 mg/ml) in 2 ml of distilled water.

The production process was defined to maintain the ratio of 1:4 between thrombin and fibrinogen by setting the following spray flows:
the fibrinogen solution was loaded into a 10-ml syringe and sprayed at a flow rate of 0.33 ml/min;
the thrombin solution was loaded into a 5-ml syringe and sprayed at a flow rate of 0.167 ml/min;
the CB-PL and PLG solution was loaded into a 5-ml syringe and sprayed at a flow rate of 0.167 ml/min.

The following production parameters were used:
mandrel diameter=3 cm;
rotation speed of the mandrel=88 rpm-translation speed of the gun-carrier carriage 13=23.3 cm/s;
extension in the axial direction of the portion of the mandrel (10) struck by the overlapping jets=5 cm;
distance of the outlet orifice of the dispensers (12) from the axial lateral surface of the mandrel (10)=4 cm;
air supply pressure to the dispensers (12) to generate the jets=12 psi.

At the end of the process, the material sprayed onto the mandrel was incubated for 1 h at 37° C. to allow complete polymerization of the fibrin (FIG. 2).

The dressing was fixed on a plastic support to avoid dimensional changes during the freeze-drying process; then the dressing and support assembly were frozen at −50° C. for about 30 min and freeze-dried for about 5 h. The freeze-dried dressing may then be reconstituted by soaking in saline solution at room temperature.

Tensile tests were then performed on specimens having the same dimensions in height and width using a universal tensile machine (Zwick Roell, Z1.0 Zwick GmbH & Co.) equipped with a 100N load cell.

Tests were performed on the fresh sample obtained from the type 1 dressing containing 10 mg of PLG. Three fibrin samples obtained by casting were considered as a comparison. The specimens were made by using a mold inside of which first the fibrinogen solution was deposited and then the thrombin solution. The specimens were allowed to cure for 15 min before being removed from the mold. The test was performed three times with a fibrin-specific tensile speed. The specimens were brought to rupture. For each specimen, the tensile force and elongation were measured and collected using the software TestExpert II (Zwick GmbH & Co.) to obtain the stress-strain graph. Specifically, the engineering effort was calculated as the ratio of the tensile force to the initial cross-sectional area of the specimen. Strain was calculated as the ratio of change in the distance between the grips to the initial distance. The elastic modulus for each specimen was calculated by considering the slope of the linear section of the stress-strain graph and then averaged over the number of specimens. The maximum stress and strain were also calculated for each sample.

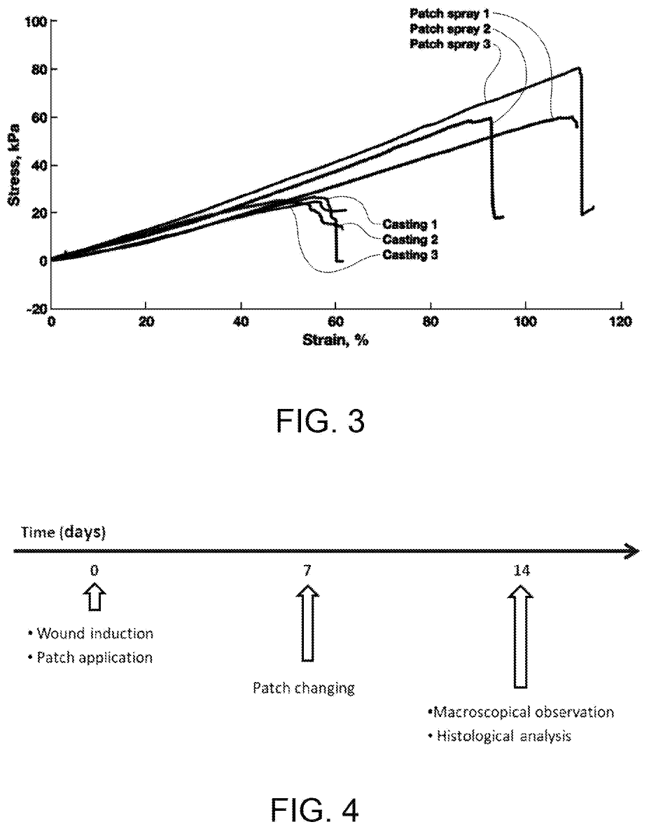

FIG. 4 shows a comparison of the stress-strain characteristics of samples extracted from fibrin-based patches manufactured with the “spray machine” equipped with 3 converging spray guns, to those from fibrin samples obtained by casting (simply mixing fibrin and thrombin until a gel is obtained, the geometry of which comes to be that of the mold in which the gel was formed).

| | Patch spray | Casting |
|---|---|---|
| Strain at rupture % [mm/mm] | 106.8 ± 10.2 | 62.2 ± 1.3 |
| Stress at rupture [kPa] | 66.6 ± 1.2 | 25.3 ± 3.1 |

As may be seen from the above table, the specimens obtained from the patch manufactured by the spray technique are characterized by a higher mean value of stress and strain at rupture than the specimens manufactured by casting. The fibrin patch manufactured by the spray technique is therefore stronger and has a more elastic behavior than the one made by casting.

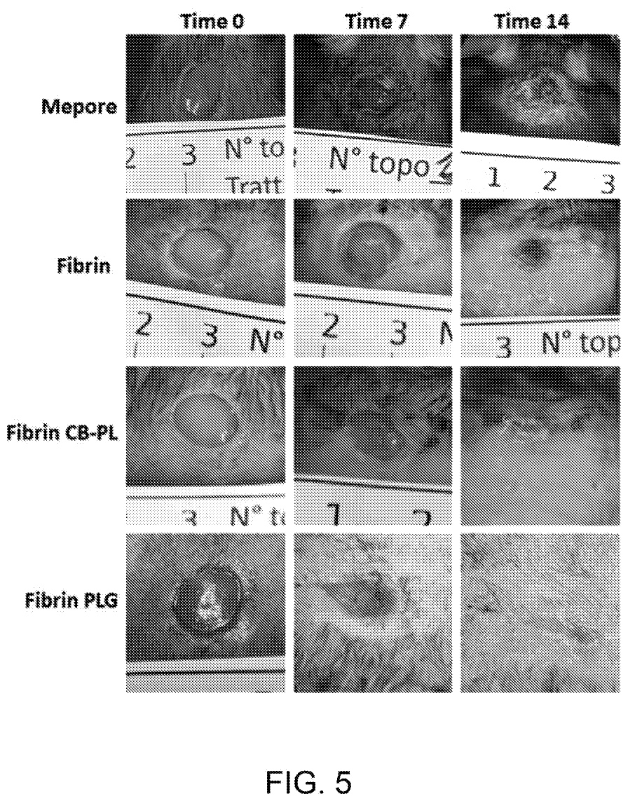

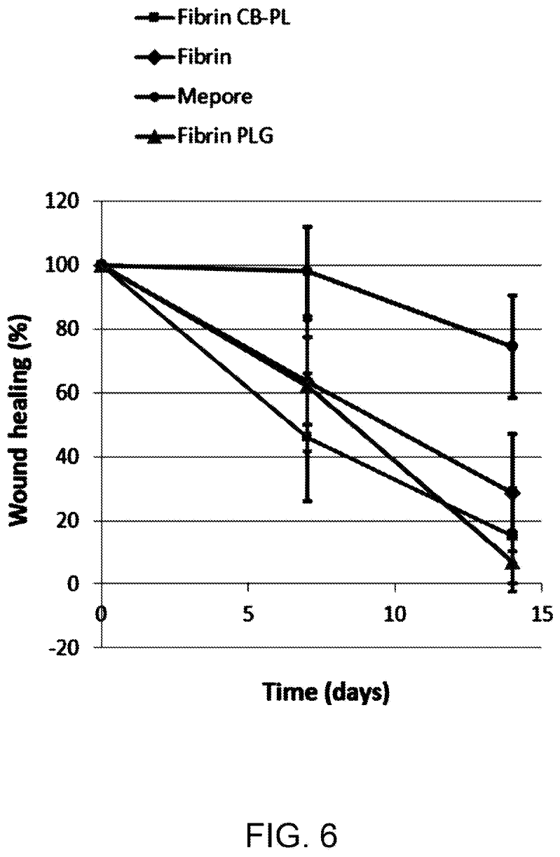

An in vivo evaluation of the effect of the patch on the healing of the wound in male diabetic mice was then performed (BKS.Cg-m+/+Lepr, db/db). Briefly, a full-thickness skin wound of 8 mm in diameter was created in each mouse, and the bioactive fibrin patch (PLG or CB-PL) or the fibrin patch as such was applied. As a control (untreated wound), Mepore® polyurethane film (Mölnlycke Health Care Srl, Göteborg, Sweden), a transparent, breath-able, self-adhesive dressing that has no bioactive properties, was used. All experimental groups were treated with a secondary dressing (Mepore®) to hold the patches in place and maintain wound sterility. At 14 days (FIG. 4), the patches were removed and the animals were sacrificed by inhalation of an overdose of isoflurane, and the wounds were photographed for macroscopic evaluation (FIGS. 5 and 6) before tissue excision and histological evaluation.

The fibrin patch loaded with PLG induced 93% of wound closure in the in vivo experiment at 14 days, the fibrin patch loaded with CB-PL 85%, and the fibrin patch as such 71%. The wound not treated with a bioactive patch (treated only with Mepore) showed 26% wound closure.

Various aspects and embodiments of a method for manufacturing a medical patch for the local and controlled release of bioactive substances for the treatment of diabetic ulcers, and a medical patch obtained by such a method according to the invention have been described. It is understood that each embodiment may be combined with any other embodiment. More-over, the invention is not limited to the embodiments described, but may be varied within the scope defined by the appended claims.

1. Method for manufacturing a medical patch for local and controlled release of bioactive substances for the treatment of ulcers, comprising the steps of:
a) providing a mandrel (10), in the form of a tubular support;
b) spraying towards the axial lateral surface of said mandrel (10) at least two separate, simultaneous and converging jets of a nebulized solution containing fibrinogen, and of a nebulized solution containing thrombin, respectively;
c) rotating the mandrel (10) around its own axis and/or orienting the aforementioned jets in such a way as to expose to said jets a predetermined circumferential portion of the axial lateral surface of said mandrel (10), until deposition on said circumferential portion of a layer of material (M) of predetermined dimensions;
d) incubating the material (M) until the fibrin contained therein is polymerized.

2. Method according to claim 1, wherein step b) is carried out by spraying towards the axial lateral surface of the mandrel (10) a third separate jet, simultaneous and converging with respect to the other two jets, of a nebulized solution containing plasminogen (PLG) and/or platelet lysate (PL) and/or metalloprotease inhibitors and/or antibiotics and/or anti-inflammatories, and/or nanoparticles charged with active ingredients and/or nanovesicles secreted by cells and containing bioactive molecules.

3. Method according to claim 2, wherein the platelet lysate (PL) is cord blood platelet lysate (CB-PL).

4. Method according to any of the preceding claims, wherein the solution containing fibrinogen contains fibrinogen in a concentration ranging from 20 mg/ml to 100 mg/ml, and the solution containing thrombin contains thrombin in a concentration ranging from 500 IU/ml to 3500 IU/ml.

5. Method according to any of claims 2 to 4, wherein the solution containing platelet lysate (PL) contains platelet lysate (PL) in a concentration ranging from 0.5×10⁹ plt/ml to 10×10⁹ plt/ml and the solution containing plasminogen (PLG) contains plasminogen (PLG) in a concentration ranging from 5 mg/ml to 100 mg/ml.

6. Method according to any of the preceding claims, wherein step b) is carried out by means of dispensers (12), each adapted to spray a respective nebulized solution in the direction of the axial lateral surface of the mandrel (10).

7. Method according to claim 6, wherein the nebulized solution containing fibrinogen is delivered from the respective dispenser (12) with a flow rate ranging from 0.1 ml/min to 0.4 ml/min, and the nebulized solution containing thrombin is delivered from the respective dispenser (12) with a flow rate ranging from 0.05 ml/min to 0.4 ml/min.

8. Method according to claim 5 or 6, wherein the nebulized solution containing platelet lysate (PL) and/or plasminogen (PLG) is delivered by the respective dispenser (12) with a flow rate ranging from 0.05 ml/min and 0.4 ml/min.

9. Method according to one of claims 5 to 8, wherein step c) is carried out by translating the dispensers (12) along a direction parallel to the axis of the mandrel (10) and/or by rotating the dispensers (12) around the mandrel axis (10).

10. Method according to any of the preceding claims, wherein the diameter of the mandrel (10) is within a range of 3-10 cm, and/or the rotational speed of the mandrel (10) is within a range of 30-120 rpm, and/or the translation speed of the dispensers (12) along a direction parallel to the axis of the mandrel (10) is within a range of 10-50 cm/s, and/or the axial extension of the portion of the mandrel (10) struck by the superimposed jets is within a range of 2-20 cm, and/or the distance of the outlet orifice of the dispensers (12) from the axial lateral surface of the mandrel (10) is within a range of 2-6 cm, and/or the air supply pressure to the dispensers (12) to generate the jets is within a range of 8-16 psi.

11. Method according to any of the preceding claims, comprising the step of freeze-drying the layer of material obtained in step d).

12. Method according to claim 11, wherein the freeze-drying step is preceded by the step of fixing the layer of the material obtained in step d) on a plastic support, and by the step of freezing the assembly thus obtained at −50° for 30 min.

13. Medical patch obtained according to the method of any of claims 1 to 12, for use in the therapeutic treatment of ulcers.

14. Medical patch for use according to claim 13, wherein the ulcers are chronic vascular or diabetic ulcers.

15. Medical patch obtained according to the method of any of claims 1 to 12, for use as a filler in the therapeutic treatment of tissue defects.

16. Apparatus for manufacturing a medical patch, comprising:
a mandrel (10), in the form of a tubular support; and
a plurality of dispensers (12), each configured to deliver a nebulized solution in such a way that the outgoing jet strikes at least part of the axial lateral surface of said mandrel (10);
wherein the mandrel (10) is rotatable around its own axis, and/or the dispensers (12) are translatable along a direction parallel to the axis of the mandrel (10) and/or rotatable around the axis of the mandrel (10).

17. Apparatus according to claim 16, wherein the dispensers (12) are each configured to deliver a flow rate of nebulized solution ranging from 0.05 ml/min to 0.4 ml/min.

18. Apparatus according to claim 16 or 17, wherein the diameter of the mandrel (10) is within a range of 3-10 cm, and/or the mandrel (10) is configured to rotate with a speed within a range of 30-120 rpm, and/or the dispensers (12) are configured to move along a direction parallel to the axis of the mandrel (10) with a speed within a range of 10-50 cm/s, and/or the dispensers (12) are configured to jointly strike with the respective jets a portion of the mandrel (10) having an axial extension within a range of 2-20 cm, and/or the distance of the outlet orifice of the dispensers (12) from the axial lateral surface of the mandrel (10) is within a range of 2-6 cm, and/or the dispensers (12) are configured to be supplied with air at a pressure within a range of 8-16 psi.
